(19) United States
(12) Patent Application Publication
(10) Pub. No.: US 2004/0034198 A1
(43) Pub. Date: Feb. 19, 2004

(54) Mutant p53 (delta126-132) protein and uses thereof

(76) Inventors: Kimberly Kline, Austin, TX (US); Bob G. Sanders, Austin, TX (US); Weiping Yu, Austin, TX (US)

Correspondence Address: Benjamin Aaron Adler, ADLER & ASSOCIATES, 8011 Candle Lane, Houston, TX, 77071, US

(21) Appl. No.: 10/444,287

(22) Filed: May 23, 2003

Related U.S. Application Data
(60) Provisional application No. 60/383,034, filed on May 24, 2002.

(51) Int. Cl. C07K 14/705; C07H 21/04; C12P 21/02; C12N 5/06

(52) U.S. Cl. 530/350; 536/23.5; 435/69.1; 435/320.1; 435/325

(57) ABSTRACT

A p53 cDNA with a 21 nucleotide base deletion that codes for a seven amino acid deleted p53 protein was disclosed herein. The mutant p53 exhibits high cellular retention and is capable of rendering tumor cells sensitive to apoptotic inducing agents such as γ-irradiation or chemotherapeutic agents. The mutant p53 protein can be delivered separately or in combination with apoptotic inducing agents via aerosol liposome/transfection/infection methods to treat cellular proliferative diseases and disorders in humans and animals.

### CROSS-REFERENCE TO RELATED APPLICATION

This non-provisional patent application claims benefit of provisional patent application U.S. Serial No. 60/383,034, filed May 24, 2003, now abandoned.

### BACKGROUND OF THE INVENTION

1. Field of the Invention

The present invention relates generally to the study of the functions and uses of p53 gene. More specifically, the present invention discloses the isolation and identification of a mutant p53 gene product that renders tumor cells sensitive to apoptotic inducing agents such as chemotherapeutic agents or γ-irradiation.

2. Description of the Related Art

Most cancers undergo increased genetic lesions and epigenetic events over time, and eventually may become highly metastatic and difficult to treat. Surgical removal of localized cancers has proven effective only when the cancer has not spread beyond the primary lesion. Once the cancer has spread to other tissues and organs, the surgical procedures must be supplemented with other more specific procedures to eradicate the malignant cells.

Most of the commonly utilized supplementary procedures for treating malignant cells such as chemotherapy or radiation are not localized to the tumor cells and, although they have a proportionally greater destructive effect on malignant cells, often affect normal cells to some extent. Moreover, a wide variety of pathological cell proliferative conditions exist for which novel therapeutic strategies and agents are needed to provide effective treatment. These pathological conditions may occur in almost all cell types capable of abnormal cell proliferation or abnormal responsiveness to cell death signals. Among the cell types that exhibit pathological or abnormal growth and death characteristics include, but are not limited to, fibroblasts, vascular endothelial cells and epithelial cells. Hence, more effective methods are highly desirable to treat local or disseminated pathological conditions in all or almost all organ and tissue systems of individuals. p 53 gene mutation is the most common tumor suppressor gene mutation found in human neoplasia (Bennett, 1999). Loss of p53 function is considered a key event in the progression of a normal cell to a cancer phenotype. Numerous p53 mutations, with subsequent loss of biological function, have been found in human cancers, and the majority of the mutations are point mutations that reside in the sequence specific DNA binding domains (Cho et al., 1994).

The prior art is deficient in methods of delivering and expressing biologically functional mutant p53 into tumor cells to provide new and novel means of prevention and treatment for pathological cell proliferative conditions. The present invention fulfills this long-standing need and desire in the art.

### SUMMARY OF THE INVENTION

The present invention discloses a mutant p53 protein that possesses the ability to sensitize tumor cells to apoptotic inducing agents. More specifically, this invention relates to the isolation and identification of a p53 cDNA (SEQ ID NO. 1) exhibiting a 21 nucleotide deletion that produces a seven amino acid deleted p53 protein (SEQ ID NO. 2) with functional properties of rendering tumor cells sensitive to apoptotic inducing agents, including chemotherapeutic agents. High cellular retention levels of this mutant p53 protein with functional attributes that render tumor cells sensitive to apoptotic inducing agents provides a promising candidate for treatment and prevention of cancers.

The cDNA sequence disclosed herein encodes a mutant p53 that has a 21 base pair deletion starting at position 376 through 396, and the deleted 21 nucleotides code for amino acids tyrosine-serine-proline-alanine-leucine-asparagine-lysine. Tyrosine and serine are potential phosphorylation sites. A schematic diagram of the mutant p53 protein showing the position of the 7 amino acid deletion (126-132) in relation to the functional domains of wild type p53 (Modified from Bennett, 1999) is presented in FIG. 1.

The present invention includes expression vectors that encode the mutant p53 protein, as well as host cells that contain these expression vectors.

The present invention is also drawn to methods of using the mutant p53 protein disclosed herein to increase a cell's sensitivity to apoptotic inducing agent or inhibit tumor cell growth.

In another aspect of the present invention, there are provided methods of using the mutant p53 protein to treat neoplastic or non-neoplastic cell proliferative diseases.

### BRIEF DESCRIPTION OF THE DRAWINGS

FIG. 1 is a schematic diagram of the mutant p53 protein showing the position of the 7 amino acid deletion (126-132) in relation to the functional domains of wild type p53. Abbreviations: N: NH2-terminal; C: COOH-terminal; I-V: conserved domains; α and β: oligomerization motifs; NLS: nuclear localization signal.

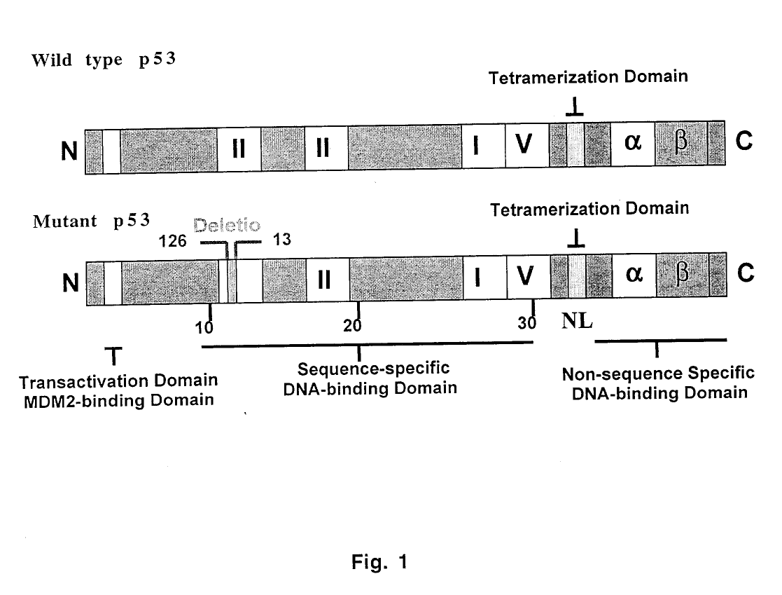

FIG. 2A shows that three c-Jun over-expressing clones (2-16, 2-31, and 2-33) exhibit high levels of c-Jun protein, high levels of p53 protein, and reduced levels of anti-apoptotic Bcl-2 and Bcl-XL protein in comparison to vector control cells (7-1, 7-2, and 7-3). Bax levels were not changed. FIG. 2B shows that the three MCF-7 clones express high levels of p53 message RNA and no Bcl-2 mRNA in comparison to three vector control cells. 18S RNA was used as an internal control.

FIG. 3 shows that MCF-7 cells stably transfected with wild type c-jun in comparison to vector control are highly sensitive to apoptotic inducing agents vitamin E succinate (VES), N-(4-hydroxyphenyl) retinamide (4-HPR), ceramide and gamma irradiation.

FIG. 4A shows a high degree of DNA fragmentation exhibited by MCF-7 c-Jun over-expressing cells cultured in the presence of vitamin E succinate, N-(4-hydroxyphenyl) retinamide, ceramide and gamma irradiation. FIG. 4B further shows DNA fragmentation as determined by DNA laddering.

FIG. 5 shows that MCF-7 cells transiently transfected with antisense oligomers to p53 exhibit reduced levels of p53 protein and increased levels of anti-apoptotic Bcl-2 protein.

FIG. 6 illustrates the process for generating pGFP, pTRE, pGST, pHIS, and pcDNA3 plasmids expressing mutant p53 and wild type p53.

FIG. 7 shows the expression of HA-tagged mutant p53 protein and HA-tagged wild type p53 protein in MCF-7 human breast cancer cells. Both wild type p53 and mutant p53 enhance the expression of p53-dependent p21(waf1/cip1), and down-regulate p53 dependent Bcl2-protein, verifying that mutant p53 retains relevant biological function.

FIG. 8 shows the expression of green fluorescent protein (GFP) in human MCF-7 cells transiently transfected with pGFP (vector control), GFP-tagged wild type p53 cDNA or GFP-mutant p53. Both wild type and mutant p53 were located in the nucleus of MCF-7 cells.

FIG. 9 shows that MCF-7 cells transiently transfected with mutant p53 (over-expressing p53) exhibit enhanced apoptosis when treated with compound #1.

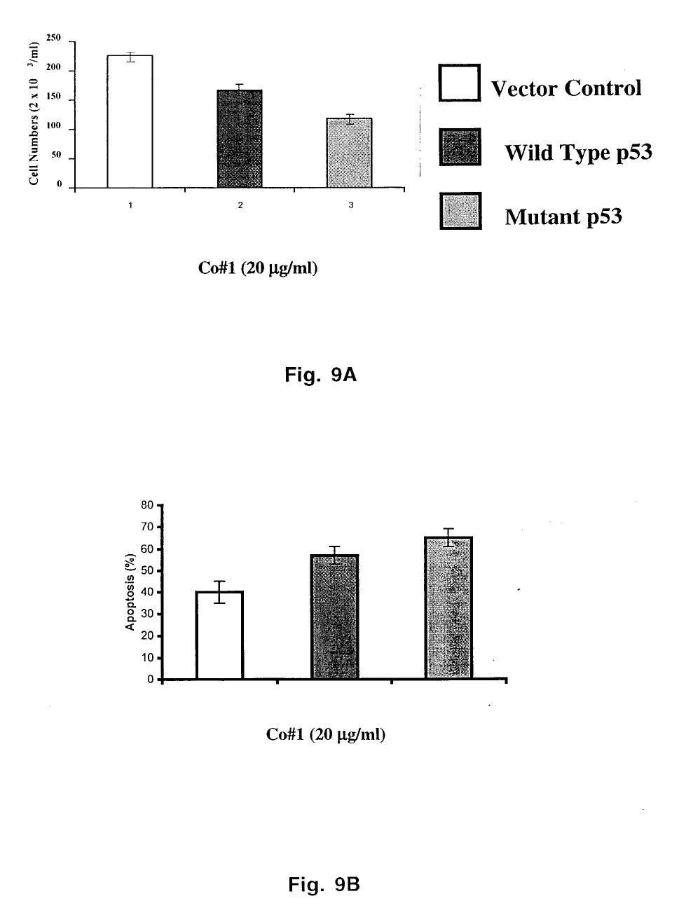

FIG. 10 shows MDA-MB-435 (FIG. 10A) and MCF-7 cells (FIG. 10B) transiently transfected with wildtype p53 or mutant p53 (D126-132) exhibit enhanced sensitivity to induction of apoptosis by α-TEA or γ-irradiation treatments.

### DETAILED DESCRIPTION OF THE INVENTION

p53, a tumor suppressor gene protein of 393 amino acids, is a transcription factor exhibiting both sequence-specific and non-specific DNA binding, and interacts with various cellular and viral proteins (Bennett, 1999). p53 is a multi-functional protein, regulating cell proliferation, cell cycle check points, growth arrest, apoptosis, and controlling the propagation of damaged DNA (reviewed by Bennett, 1999). P53 protein has been divided into five domains that are conserved among species: domain I, N-terminal activation domain; domains II-IV, core domains mediating sequence specific DNA binding; and domain V, carboxyl-terminal domain with tetramerization functions (Cho et al., 1994; Soussi and May, 1996; Prives and Hall, 1999). Numerous p53 mutations with loss of biological function have been found in human cancers, and the majority of the mutations are point mutations that reside in sequence specific DNA binding domains (Cho et al., 1994).

The p53 mutant described in this disclosure has a seven amino acid deletion in the fifth exon in domain II involving amino acid residues 126-132 (tyrosine-serine-proline-alanine-leucine-asparagine-lysine). Tyrosine and serine are two potential phosphorylation sites that have been deleted in this mutant p53 protein. The p53 deletion is located in a region in loop 1 of the p53 protein that is structurally described as the “S2-S2′ B hairpin” (amino acid residues 124-141), a region that is thought to provide framework for orientation of the DNA binding region (Cho et al., 1994).

A search of the p53 literature shows that mutant p53(Δ126-132) was reported in MCF-7 cells expressing high levels of c-Jun (O'Connor et al., 1997). These researchers conducted functional studies using the c-jun over-expressing cells and found a lack of response to induction of a p53-dependent gene, inability to induce G1 cell cycle arrest in response to gamma irradiation, and inability to activate gamma irradiation inducible genes. Hence, based on the National Cancer Institute anticancer Drug Screen, these researchers concluded that mutant p53(Δ126-132) was non functional. However, as described below, the present invention demonstrates positive functional results with mutant p53 (Δ126-132). More specifically, the mutant p53 of the present invention possesses the ability to sensitize tumor cells to apoptotic inducing agents. In contrast to other mutant p53 proteins that may act as dominant negative mutants with the property of inhibiting the function of wild type p53, the mutant p53 described herein maintains biological functions that render cells sensitive to apoptotic inducing agents. This anti-tumor activity of sensitizing tumor cells to the induction of apoptosis suggests that the p53 mutant disclosed herein may be a promising candidate for uses in the treatment and prevention of cancers.

As used herein, the terms “mutant p53”, “mutant p53 constructs”, and “mutant p53 antitumor functions” shall include the expression and analyses of mutant p53 and constructs in vitro and in vivo.

As used herein, the term “individual” shall refer to animals and humans.

As used herein, the term “biologically inhibiting” or “inhibition” of the growth of syngenic tumor grafts shall include partial or total growth inhibition and also is meant to include decreases in the rate of proliferation or growth of tumor cells. The biologically inhibitory dose may be determined by assessing the effects of the test element on malignant or abnormally proliferating cell growth in tissue culture, tumor growth in animals or any other method known to those of ordinary skill in the art.

As used herein, the term “inhibition of metastases” shall include partial or total inhibition of tumor cell migration from primary site to other organs. The biological metastatic inhibitory dose may be determined by assessing the effects of the test element on malignant or abnormally proliferating cell growth in tissue culture, tumor growth in animals or any other method known to those of ordinary skill in the art.

As used herein, the term “inhibition of angiogenesis” shall include partial or total inhibition of tumor blood vessel formation or reduction in blood carrying capacity of blood vessels supplying blood to tumors.

As used herein, the term “induction of programmed cell death or apoptosis” shall include partial or total cell death with cells exhibiting established morphological and biochemical apoptotic characteristics. The dose that induces apoptosis may be determined by assessing the effects of the test element on malignant or abnormally proliferating cell growth in tissue culture, tumor growth in animals or any other method known to those of ordinary skill in the art.

As used herein, the term “induction of DNA synthesis arrest” shall include growth arrest due to blockages in GO/G1, S, or G2/M cell cycle phases. The dose that induces DNA synthesis arrest may be determined by assessing the effects of the test element on malignant or abnormally proliferating cell growth in tissue culture, tumor growth in animals or any other method known to those of ordinary skill in the art.

As used herein, the term “induction of cellular differentiation” shall include growth arrest due to treated cells being induced to undergo cellular differentiation as defined by established morphological and biochemical differentiation characterization, a stage in which cellular proliferation does not occur. The dose that induces cellular differentiation may be determined by assessing the effects of the test element on malignant or abnormally proliferating cell growth in tissue culture, tumor growth in animals or any other method known to those of ordinary skill in the art.

In accordance with the present invention there may be employed conventional molecular biology, microbiology, and recombinant DNA techniques within the skill of the art. Such techniques are explained fully in the literature. See e.g., Maniatis, Fritsch & Sambrook, “Molecular Cloning: A Laboratory Manual (1982); “DNA Cloning: A Practical Approach,” Volumes I and II (D.N. Glover ed. 1985); “Oligonucleotide Synthesis” (M. J. Gait ed. 1984); “Nucleic Acid Hybridization” [B. D. Hames & S. J. Higgins eds. (1985)]; “Transcription and Translation” [B. D. Hames & S. J. Higgins eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

The present invention includes expression vectors that encode the mutant p53 protein, as well as host cells that contain these expression vectors. The claimed vectors comprise in operable linkage: an origin of replication; a promoter; and a DNA of SEQ ID NO. 1 coding for the mutant p53 protein of SEQ ID NO. 2. The vector may further comprise sequence encoding a tag linked to the mutant p53 protein. In general, the protein tag can be a HA tag, a green fluorescent protein tag, a GST tag or a HIS tag.

A “vector” may be defined as a replicable nucleic acid construct, e.g., a plasmid or viral nucleic acid. Vectors may be used to amplify and/or express nucleic acid encoding the mutant p53 disclosed herein. An “expression vector” is a replicable construct in which a nucleic acid sequence encoding a polypeptide is operably linked to suitable control sequences capable of effecting expression of the polypeptide in a cell. The need for such control sequences will vary depending upon the cell selected and the transformation method chosen. Generally, control sequences include a transcriptional promoter and/or enhancer, suitable mRNA ribosomal binding sites, and sequences which control the termination of transcription and translation. Methods which are well known to those skilled in the art can be used to construct expression vectors containing appropriate transcriptional and translational control signals. See for example, the techniques described in Sambrook et al., 1989, Molecular Cloning: A Laboratory Manual (2nd Ed.), Cold Spring Harbor Press, N.Y.

A gene and its transcription control sequences are defined as being “operably linked” if the transcription control sequences effectively control the transcription of the gene. Vectors of the invention include, but are not limited to, plasmid vectors and viral vectors. Preferred viral vectors of the invention are those derived from retroviruses, adenovirus, adeno-associated virus, SV40 virus, or herpes viruses.

The present invention also includes host cells transfected with the vector described herein. As used herein, the term “host” is meant to include not only prokaryotes but also eukaryotes such as yeast, plant and animal cells. A recombinant DNA molecule or gene which encodes the mutant p53 protein of the present invention can be used to transform a host using any of the techniques commonly known to those of ordinary skill in the art. Prokaryotic hosts may include E. coli, S. tymphimurium, Serratia marcescens and Bacillus subtilis. Eukaryotic hosts include yeasts such as Pichia pastoris, mammalian cells and insect cells.

In another aspect of the present invention, there are provided a method of increasing a cell's sensitivity to apoptotic inducing agent and a method of inhibiting tumor cell growth by expressing in the cell the p53 mutant protein disclosed herein. In general, apoptotic inducing agent includes 9-nitro-camptothecin, doxorubicin, taxol or γ-irradiation. The p53 mutant protein would inhibit tumor cell growth by inducing apoptosis, DNA synthesis arrest, cell cycle arrest or cellular differentiation.

In another embodiment, there are provided methods of using the mutant p53 protein to treat cell proliferative diseases caused by neoplastic or non-neoplastic disorders in an individual. The mutant p53 can be delivered to an individual alone or in combination with other anti-cancer agents by transient transfections, infections, or aerosol liposome. In general, anti-cancer agents include γ-irradiation and chemotherapeutic agents.

Representative examples of neoplastic diseases include ovarian cancer, cervical cancer, endometrial cancer, bladder cancer, lung cancer, breast cancer, prostate cancer, testicular cancer, gliomas, fibrosarcomas, retinoblastomas, melanomas, soft tissue sarcomas, osteosarcomas, colon cancer, carcinoma of the kidney, pancreatic cancer, basal cell carcinoma, and squamous cell carcinoma.

Representative examples of non-neoplastic diseases include psoriasis, benign proliferative skin diseases, ichthyosis, papilloma, restinosis, scleroderma and hemangioma, and leukoplakia.

Methods of the present invention may also be used to treat non-neoplastic diseases that develop due to failure of selected cells to undergo normal programmed cell death or apoptosis. Representative examples of diseases and disorders that occur due to the failure of cells to die are autoimmune diseases. Autoimmune diseases are characterized by immune cell destruction of self cells, tissues and organs. A representative group of autoimmune diseases includes autoimmune thyroiditis, multiple sclerosis, myasthenia gravis, systemic lupus erythematosus, dermatitis herpetiformis, celiac disease, and rheumatoid arthritis. However, this invention is not limited to autoimmunity, but includes all disorders having an immune component, such as the inflammatory process involved in cardiovascular plaque formation, or ultra violet radiation induced skin damage.

Methods of the present invention may also be used to treat disorders and diseases that develop due to viral infections. Representative examples of diseases and disorders that occur due to viral infections include those that are caused by human immunodeficiency viruses (HIV). Since the mutant p53 disclosed herein sensitizes cells to apoptotic inducing agents that induces cell death by initiating intracellular apoptotic signaling networks, this invention has the capacity to impact signal transduction of a number of external cellular signals such as cytokines, viruses, bacteria, toxins, heavy metals, etc.

In a preferred embodiment of the present invention, the vector encoding the mutant p53 protein is administered to an individual in the form of an aerosolized liposome. A representative liposome includes, but is not limited to, a lipsome formulated with dilauroylphosphatidylcholine and the aerosol may comprise about 5% to 7.5% carbon dioxide. More particularly, the aerosol may have a ratio of polyethylenimine nitrogen to DNA phosphate (nitrogen:phosphate) from about 5:1 to about 20:1. Generally, this method may be used to inhibit tumor cell growth by apoptosis, DNA synthesis arrest, cell cycle arrest, or cellular differentiation.

In another embodiment of this method, it may further comprise a step of administering an anti-cancer agent before or after administering the vector encoding the mutant p53. Representative anti-cancer agents include 9-nitrocamptothecin, paclitaxel, doxorubicin, 5-fluorouracil, mitoxantrone, vincristine, cisplatin, epoposide, tocotecan, tamoxifen, carboplatin and γ-irradation. The anti-cancer drug can be administered in the form of an aerosolized liposome. Optionally, the vector and the anti-cancer drug are administered concurrently in the form of an aerosolized liposome as described above.

The methods of the present invention may be used to treat any animal. Most preferably, the methods of the present invention are useful in humans. Generally, to achieve pharmacologically efficacious cell killing and anti-proliferative effects, mutant p53 may be administered in any therapeutically effective dose, i.e., amounts that eliminate or reduce tumor burden and/or cell proliferation.

The following examples are given for the purpose of illustrating various embodiments of the invention and are not meant to limit the present invention in any fashion. The present examples, along with the methods, procedures, treatments, molecules, and specific compounds described herein are presently representative of preferred embodiments. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

Cloning and Expression of p53 Mutant (Δ126-132)

Mutant p53 (Δ126-132) cDNA was isolated from human MCF-7 cells stably transfected with wild type transcription factor c-jun and expressing high levels of c-Jun protein. The c-Jun over-expressing MCF-7 cells were obtained from Drs. Michael Birrer (National Institutes of Health, National Cancer Institute, Rockville, Md.) and Paul Brown (Baylor College of Medicine, Houston, Tex.). A description of the c-Jun over-expressing MCF-7 cells can be found in Yang et al. (1997) and Smith et al. (1999).

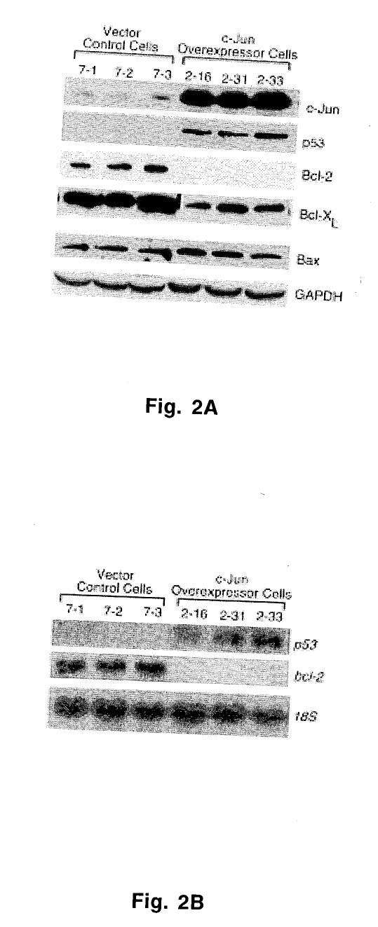

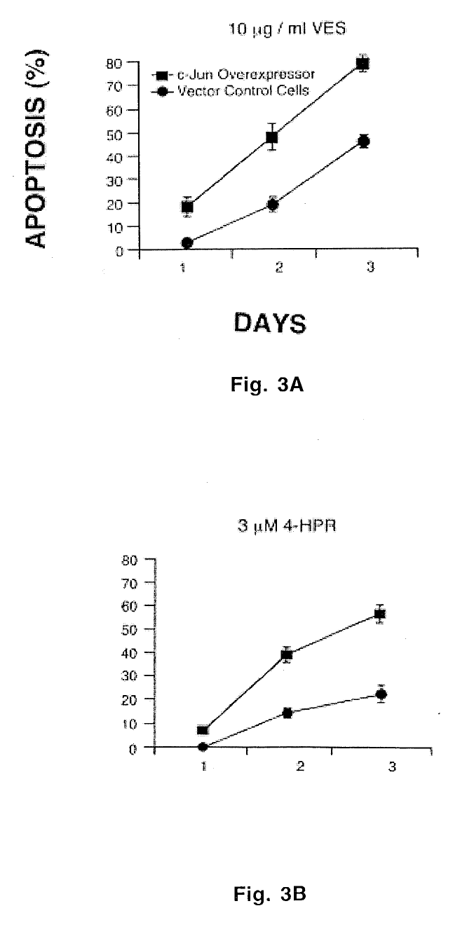

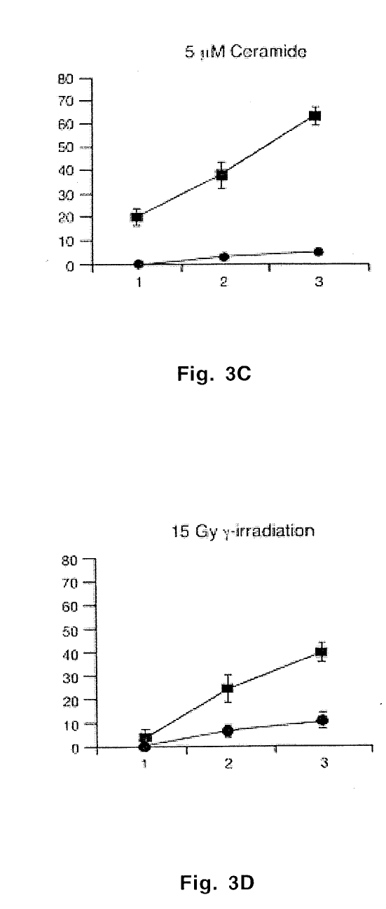

MCF-7 c-Jun over-expressing cells constitutively expressed high levels of p53 but reduced levels of Bcl-2 and Bcl-XL compared to parental vector control cells. Bax levels were not altered (FIG. 2A). At the transcription level, MCF-7 cells over-expressing c-Jun showed p53 mRNA levels to be constitutively expressed, whereas bcl-2 mRNA levels was reduced (FIG. 2B). These c-Jun over-expressing cells were highly sensitive to apoptotic inducing agents vitamin E succinate (VES), N-(4-hydroxyphenyl) retinamide (4-HPR), ceramide and gamma irradiation (FIG. 3) and exhibit high degree of DNA fragmentation when cultured in the presence of these apoptotic inducing agents (FIGS. 4A-B)

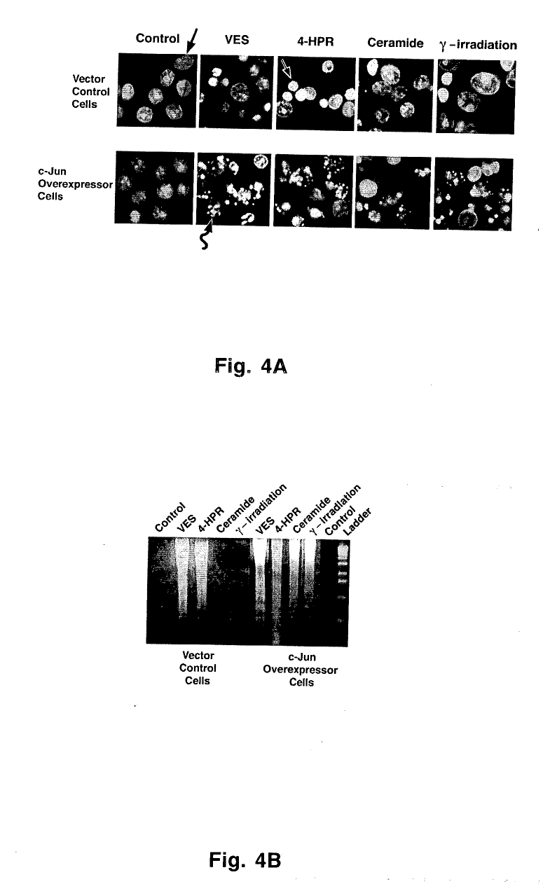

Blockage of p53 using p53 antisense oligomers in c-Jun over-expressing cells resulted in up-regulation of Bcl-2 protein, showing that p53 is regulating the expression of Bcl-2 protein (FIG. 5). Furthermore, cells treated with p53 antisense oligomers were resistant to apoptotic inducing agents (Table 1), and exhibited reduced levels of p53 protein and enhanced levels of Bcl-2 protein (FIG. 5), indicating that p53-mediated reduced levels of Bcl-2 are associated with increased sensitivity of these cells to apoptotic agents. Taken together, these data suggest that p53 in these c-Jun over-expressing cells can enhance apoptotic actions of apoptotic inducing compounds. Subsequently, cDNA cloning and nucleotide sequencing in these c-jun over-expressing cells led to the identification of a mutant p53 (Δ126-132) as described below.

TABLE 1
Effects of Antisense Oligomers to p53 on Induction of Apoptosis

| Oligomer | Induction of Apoptosis (%) Following Treatments | | | |
|---|---|---|---|---|
| Transient | With Apoptotic Agents^{b} | | | |
| Transfections^{a} | VES | 4-HPR | g-Irradiation | Ceramide |
| Antisense | 25 ± 4.5 | 17 ± 2.1 | 18 ± 3.6 | 21 ± 2.1 |
| Sense | 49 ± 3.5 | 36 ± 2.1 | 29 ± 4.0 | 39 ± 4.0 |
| Decrease (%) | 49% | 53% | 38% | 46% |

^{a} c-Jun over-expressing cells (2-31) were transiently transfected with p53 antisense or sense oligomers for 4 hours. Next, the c-Jun over-expressing cells were treated with 10 μg/ml of vitamin E succinate (VES), 3 μM of N-(4-hydroxyphenyl) retinamide (4-HPR), or 5 μM of ceramide for 2 days or 15 Gy of γ-irradiation for 3 days. The data presented are average±standard deviation of three separate experiments. ^{b}Apoptosis was determined by DAPI staining. ^{c}Percent apoptosis (increase or decrease) were determined by comparing the levels of apoptosis for each treatment group of cells transfected with antisense oligomers versus sense oligomers.

The coding area of the cDNA for human mutant p53 (Δ126-132) was amplified by RT-PCR using total RNA from MCF-7 (clone 2-31) cell line stably transfected with transcription factor c-Jun. Total RNA was extracted using RNasy Mini Kit (Qiagen). RT-PCR was performed with Superscript II RT (GIBCOBRL) using random primers. PCR was performed with the ProofStart DNA Polymerase (Qiagen). The p53 oligonucleotide primers were synthesized based on published p53 sequence (Genbank Accession #X02469) with sense oligomer primer (5′-ATG GAG GAG CCG CAG TCA GAT-3′, SEQ ID NO. 3) and antisense oligomer primer (5′-TCA GTC TGA GTC AGG CCC TTC-3′, SEQ ID NO. 4) (Integrated DNA Technologies, Inc IDT).

Five μg total RNA and random primer (GIBCOBRL) were denatured at 65° C. for 5 minutes, reverse transcribed at 42° C. for 50 min and inactivated at 70° C. for 15 minutes. Five μl of RT product then underwent 35 cycles of PCR as follows: 94° C. for 30 seconds, 55° C. for 1 minute and 72° C. for 1 minute. An approximately 1.2 kb PCR product was purified with QIAquick Gel Extraction Kit (Qiagen) and subcloned into the pGEM-T easy vector (Promega) after performing an A-tailing procedure (Promega). The construct was transformed into JM101 competent cells using hot shock. Clones were sequenced using M13 forward and reverse oligomer primers (Integrated DNA Technologies, Inc). The 1.2 kb PCR products were also sequenced with sense and antisense oligomer primers as mentioned above. The cDNA sequence and the predicted amino acid sequence for mutant p53 (Δ126-132) are shown in SEQ ID NOs. 1 and 2 respectively.

For protein expression of mutated and wild type p53, a construct containing an HA-tag on the N-terminal site was designed. The sense primer for the PCR encoded an EcoRI restrict enzyme cutting site, starting codon, HA residue, and p53 sequence from 4-21 nucleotide bases (5′-CGC GAA TTC ATG TAT GAT GTT C TAT GCT AGC CTC GAG GAG CCG CAG TCA GAT CCT, SEQ ID NO. 5). The antisense primer contained a BamHI restrict enzyme cutting site and stop codon of p53 (antisense, 5′ CGC GGA TCC TCA GTC TGA GTC AGG CCC TTC, SEQ ID NO. 6). The cloned mutant p53 (pGEM-p53-2-31 clone 1) and wild type p53 (pGEM-p53-7-2 clone 3) were used as templates. The resulting PCR mutant and wild type p53 products were subcloned into the pGEM vector for sequence analyses.

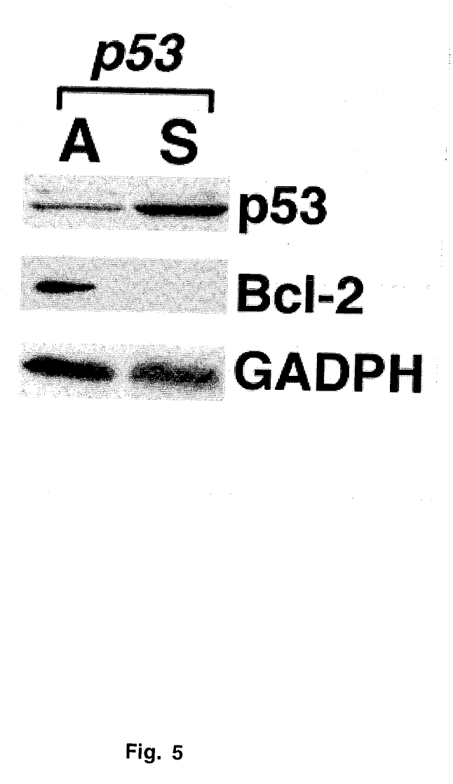

To obtain pTRE-mutant and wild type p53 on an inducible promoter, the HA-mutant and wild type p53 cDNA in pGEM were subcloned into pTRE vectors with EcoRI/BamHI cutting. The process for generating pGFP, PTRE, pGST, pHIS, and pcDNA3 plasmids expressing mutant p53 and wild type p53 is illustrated in FIG. 6.

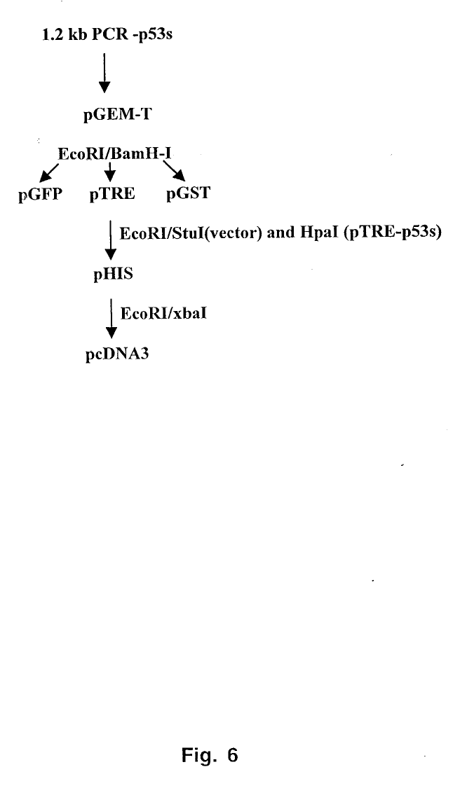

Mutant p53 can be expressed in a number of cell lines. For example, MCF-7 human breast cancer cells can be stably transfected with pTRE-HA-mutant and wild type p53 vectors. Positive clones expressing mutant and wild type p53 can be selected by screening with HA-tag antibody. MCF-7 cells can also be transiently transfected with pcDNA-3 HA-mutant and wild type p53 vectors. Mutant p53 is effective in up-regulating p21 and down-regulating Bcl-2 in transfected cells (FIG. 7).

MCF-7 cells transiently transfected with antisense oligomers to p53 exhibit increased Bcl-2 protein and loss of sensitivity to apoptotic inducing agents, providing further evidence that mutant p53 is rendering cells more sensitive to apoptotic inducing agents by regulating Bcl-2 protein levels (FIG. 5). Furthermore, over-expression of mutant p53 enhanced the ability of compound #1 to induce apoptosis providing further proof that mutant p53 exhibits relevant biology.

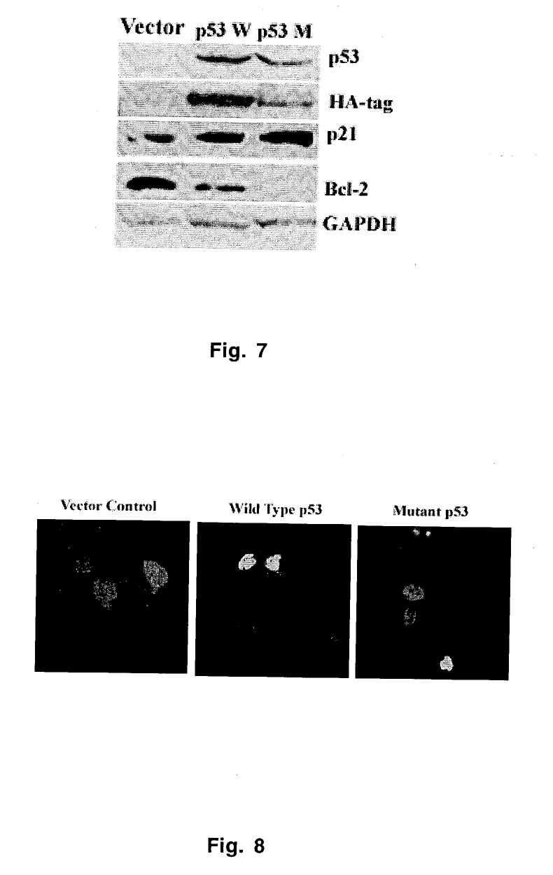

Mutant and wild type p53 can also be fused to green fluorescent protein (GFP), and GFP-tagged mutant p53 (as well as wild type p53) retains function in that mutant p53-GFP fusion protein translocated from the cytoplasm to the nucleus (FIG. 8).

### EXAMPLE2

p53 Mutant (Δ126-132) Enhances Apoptosis Induced by γ-Irradition

MDA-MB-435 (p53^{-/-}) estrogen non-responsive human breast cancer cells and MCF-7 (p53^{+/+}) estrogen responsive human breast cancer cells were transiently transfected with pcDNA vector, pcDNA-wild-type p53 or pcDNA mutant p53 (Δ126-132) constructs. Following transfection, the transfected cells were untreated or treated with 10 ug/ml α-TEA or 20 kG of γ-irradiation. Next, the cells were cultured for 2 days, and apoptosis was evaluated by nuclei staining by DAPI.

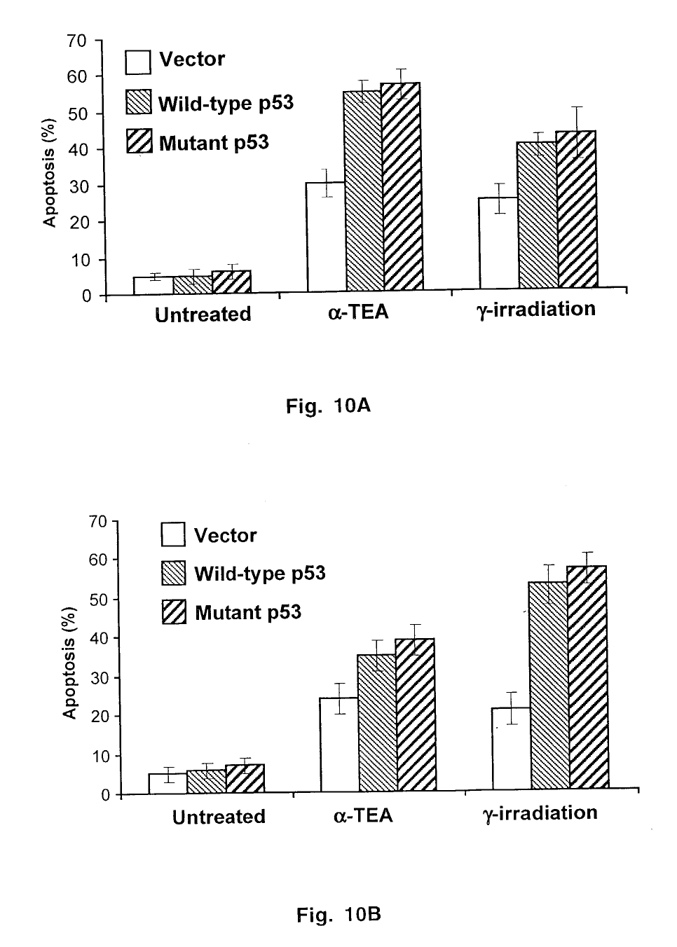

MDA-MB-435 and MCF-7 human breast cancer cells transiently transfected with either wild-type p53 or mutant p53 were more sensitive to induction of apoptosis induced by γ-irradiation or α-TEA when compared to untreated transfected cells (FIG. 10). The percent increase in apoptosis in comparison to untreated transfected cells are summarized in Table 2. These data show that mutant p53 (Δ126-132) retains function, in that it behaves similarly to wild-type p53 in providing enhanced sensitivity to induction of apoptosis by two therapeutic agents, α-TEA and γ-irradiation.

TABLE 2
Wild-type p53 And Mutant p53 (Δ126-132) Have The Ability To
Enhance Sensitivity To Induction Of Apoptosis

| | Enhanced Sensitivity (increased apoptosis %)* | | | |
|---|---|---|---|---|
| | a-TEA | | g-irradiation | |
| | MDA-MB-435 | MCF-7 | MDA-MB-435 | MCF-7 |
| Mutant p53 | 90 | 54 | 72 | 170 |
| Wild-type p53 | 83 | 46 | 64 | 150 |

*Compared to a-TEA and g-irradiation treated MDA-MB-435 and MCF-7 vector control cells.

### EXAMPLE3

In Vivo Potential for Human Cancer Cells

The mutant p53 of the present invention may be used as a therapeutic agent. Tumor growth and metastasis can be studied by ectopically or orthotopically transplanting human tumor cells into immune compromised animals such as immune compromised nude mice or severe combined immunodeficient (SCID) mice. Alternatively, in vivo studies employing well recognized animal models can be conducted. Inhibition of growth of human tumor cells transplanted into immune compromised mice provides pre-clinical data for clinical trials. In one aspect of the present invention, in vivo studies are focused on the metastatic potential of non-estrogen responsive MDA-MB-435 human breast cancer model, and a murine syngenic 66cl.4-GFP mammary cancer model.

MDA-MB-435 Breast Cancer Model:

Pathogen free Green fluorescent protein (GFP)-MDA-MB-435 FL human breast cancer cells, a highly metastatic cell line isolated from the lungs of nude mice, stably transfected with the marker protein GFP are grown as solid tumor in immune compromised nude mice. 1×10⁶ tumor cells can be orthotopically injected into the mammary fat pad or ectopically injected near the 4th and 5th nipples of female nude mice. Tumor growth, metastasis, and death of the animals are then determined. Tumor growth can be measured by caliper evaluations of tumor size. At the time of sacrifice, tumors are removed for volume measurement and histochemical examination. Organs such as spleen, lymph nodes, lungs, and bone marrow can be examined for metastatic cells by histochemical staining of tissue sections for expression of the marker green fluorescence protein.

Murine Syngenic 66cl.4-GFP Mammary Cancer Model

Pathogen free 66cl.4-GFP mammary cancer cells of Balb/c origin (100,000 to 200,000 cells) can be injected near the 4th and 5th nipples of female Balb/c mice. Tumor metastases to lungs occur in 100% of the mice. Tumor growth, metastasis, and death of the animals can be determined as described above. Tumor growth is measured by caliper evaluations of tumor size. At the time of sacrifice, tumors are removed for volume measurement and histochemical examination. Organs such as spleen, lymph nodes, lungs, and bone marrow can be examined for metastatic cells by histochemical staining of tissue sections for expression of the marker green fluorescence protein.

### EXAMPLE 4

Aerosol Liposome Preparation of Mutant p5.3 Plasmid DNA and Administration

The liposome formulation of mutant p53 plasmid DNA can be produced separately or in combination with other apoptotic inducing agents using polyethyleneimine according to the liposome/plasmid DNA procedures outlined in Densmore et al. (2001). Apoptotic inducing agents include but are not limited to vitamin E compound #1 [2,5,7,8-tetramethyl-(2R-(4R,8R, 12-trimethyltridecycl) chroman-6-yloxy) acetic acid], 9-nitro-camptothecin, doxorubicin, and taxol.

Aerosol liposome/mutant p53 plasmid DNA preparation, produced separately or in combination with apoptotic inducing agents, can be administered to tumor bearing and non-tumor bearing Balb/c mice in a sealed plastic cage. An air compressor (EZ-Air PM 15F, Precision Medical) producing 10L/min airflow can be used with an Aero Mist nebulizer (CIS-US, Inc. Bedford, Mass.) to generate aerosol particles. The preparations are reconstituted by bringing the liposomes to room temperature before adding enough distilled water to bring the final volume to 5 mls. The solution is allowed to swell at room temperature for 30 minutes with periodic inversion and then added to the nebulizer. The nebulizer can be connected via accordian tubing (1 cm inside diameter) to an entry in one end of the cage. Aerosol will be discharged through an opening at the opposite end of the cage. For safety, nebulizing will be done in a hood. Aerosol is administered to the mice in a closed container cage until all treatment is gone (approximately 30 minutes for delivery of total volume of 5 mls).

The following references were cited herein:

Bennet., Biochem. Pharmacol. 58: 1089-1095 (1999).

Densmore et al., Cancer Gene Therapy 8: 619-627 (2001).

O'Connor et al., Cancer Research 57:4285-4300 (1997).

Prives, Cell 78: 543-546 (1994).

Prives and Hall, J. Pathology 187:112-126 (1999).

Smith et al., Oncogene 18: 6053-6070 (1999).

Soussi and May, J. Molecular Biology 260:623-637 (1996).

Yang et al., Cancer Research 57: 4652-4661 (1997).

Yunje et al., Science 265: 346-355 (1994).

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

What is claimed is:

1. A vector comprising:
(a) an isolated DNA of SEQ ID NO. 1 or an isolated DNA differing from SEQ ID NO. 1 in codon sequence due to degeneracy of the genetic code, wherein said DNA encodes a mutant p53 protein of SEQID NO. 2; and
(b) regulatory elements necessary for expressing said DNA in a cell.

2. The vector of claim 1, wherein said vector comprises sequence encoding a tag linked to said mutant p53 protein.

3. The vector of claim 2, wherein said tag is selected from the group consisting of a HA tag, a green fluorescent protein tag, a GST tag and a HIS tag.

4. A host cell transfected with the vector of claim 1.

5. The host cell of claim 4, wherein said cell is selected from the group consisting of bacterial cells, mammalian cells, plant cells and insect cells.

6. A method of increasing a cell's sensitivity to an apoptotic inducing agent, comprising the step of administering to said cell an expression vector comprising an isolated DNA of SEQ ID NO. 1 or an isolated DNA differing from SEQ ID NO. 1 in codon sequence due to degeneracy of the genetic code, wherein expression of mutant p53 protein encoded by said vector increases the cell's sensitivity to apoptotic inducing agent.

7. The method of claim 6, wherein said apoptotic inducing agent is selected from the group consisting of 9-nitro-camptothecin, doxorubicin, taxol and γ-irradiation.

8. A method of inhibiting tumor cell growth, comprising the step of administering to said tumor cell an expression vector comprising an isolated DNA of SEQ ID NO. 1 or an isolated DNA differing from SEQID NO. 1 in codon sequence due to degeneracy of the genetic code, wherein expression of mutant p53 protein encoded by said vector inhibits the growth of said tumor cell.

9. The method of claim 8, wherein said mutant p53 protein inhibits tumor cell growth by inducing an effect selected from the group consisting of apoptosis, DNA synthesis arrest, cell cycle arrest and cellular differentiation.

10. A method for the treatment of cell proliferative diseases in an individual, comprising the step of administering to said individual an expression vector comprising an isolated DNA of SEQ ID NO. 1 or an isolated DNA differing from SEQ ID NO. 1 in codon sequence due to degeneracy of the genetic code, wherein expression of the mutant p53 protein encoded by said vector provides treatment for cell proliferative diseases in said individual.

11. The method of claims 10, wherein said vector is administered in the form of an aerosolized liposome.

12. The method of claim 11, wherein said liposome comprises dilauroylphosphatidylcholine.

13. The method of claim 11, wherein said liposome comprises about 5% to 7.5% carbon dioxide.

14. The method of claim 11, wherein said liposome has a ratio of polyethylenimine nitrogen to DNA phosphate (nitrogen:phosphate) from about 5:1 to about 20:1.

15. The method of claim 10, further comprising the step of administering γ-irradiation or an anti-cancer compound before or after administering said vector.

16. The method of claim 15, wherein said anti-cancer compound is selected from the group consisting of 9-nitrocamptothecin, paclitaxel, doxorubicin, 9-nitrocamptothecin, 5-fluorouracil, mitoxantrone, vincristine, cisplatin, epoposide, tocotecan, tamoxifen, and carboplatin.

17. The method of claim 15, wherein said anti-cancer compound is administered in the form of an aerosolized liposome.

18. The method of claim 15, wherein said vector and said anti-cancer compound are administered concurrently or sequentially in the form of an aerosolized liposome.

19. The method of claim 18, wherein said liposome comprises dilauroylphosphatidylcholine.

20. The method of claim 18, wherein said liposome comprises about 5% to 7.5% carbon dioxide.

21. The method of claim 18, wherein said liposome has a ratio of polyethylenimine nitrogen to DNA phosphate (nitrogen:phosphate) from about 5:1 to about 20:1.

22. The method of claims 10, wherein said cell proliferative disease is selected from the group consisting of neoplastic diseases and non-neoplastic disorders.

23. The method of claim 22, wherein said neoplastic disease is selected from the group consisting of ovarian cancer, cervical cancer, endometrial cancer, bladder cancer, lung cancer, breast cancer, testicular cancer, prostate cancer, gliomas, fibrosarcomas, retinoblastomas, melanomas, soft tissue sarcomas, osteosarcomas, leukemia, colon cancer, carcinoma of the kidney, pancreatic cancer, basal cell carcinoma, and squamous cell carcinoma.

24. The method of claim 22, wherein said non-neoplastic disease is selected from the group consisting of psoriasis, benign proliferative skin diseases, ichthyosis, papilloma, restinosis, scleroderma, hemangioma, leukoplakia, viral diseases, inflammatory process and autoimmune diseases.

25. The method of claim 24, wherein said autoimmune disease is selected from the group consisting of autoimmune thyroiditis, multiple sclerosis, myasthenia gravis, systemic lupus erythematosus, dermatitis herpetiformis, celiac disease, and rheumatoid arthritis.

26. The method of claim 24, wherein said viral disease is caused by Human Immunodeficiency Virus.

27. The method of claim 24, wherein said inflammatory process is selected from the group consisting of inflammatory processes involved in cardiovascular plaque formation and ultraviolet radiation induced skin damage.
